(19) United States
(12) Patent Application Publication
(10) Pub. No.: US 2021/0244865 A1
(43) Pub. Date: Aug. 12, 2021

(54) BREAST PUMP DEVICE

(71) Applicant: KONINKLIJKE PHILIPS N.V., EINDHOVEN (NL)

(72) Inventors: Christoph DOBRUSSKIN, NUENEN (NL); Daan Hendrik GOSENSHUIS, WAALRE (NL); Coen Petrus Martinus CLAASSEN, LOMMEL (BE); Eyob Atnafu AMRA, EINDHOVEN (NL)

(21) Appl. No.: 17/271,225

(22) PCT Filed: Sep. 11, 2019
(86) PCT No.: PCT/EP2019/074148

§ 371 Date: Feb. 25, 2021

(30) Foreign Application Priority Data
Sep. 12, 2018 (EP) 18194081.8

(51) Int. Cl. A61M 1/06

(52) U.S. Cl. CPC A61M 1/062; A61M 2205/13; A61M 2205/3331; A61M 2205/15; A61M 2205/583; A61M 2205/581; A61M 2205/70

(57) ABSTRACT

A breast pump device (100) is provided with a breast receiving portion (130), a pressure source (120) for generating an over- or under pressure, a controller (110) and a breast detector (140) for detecting a first state when no breast is in the breast receiving portion and a second state when a breast is in the breast receiving portion (130). The controller (110) changes a parameter of the breast pump device and/or outputs a notification if the breast detector (140) has detected the second state. The breast detector (140) detects the first or second state based on operating parameters of the pressure source (120). The controller (110) operates the pressure source (120) in a first operating mode if the breast detector (140) has detected the first state or in a second operating mode if the breast detector (140) has detected the second state.

### FIELD OF THE INVENTION

The invention relates to a breast pump device and a method of operating a breast pump device.

### BACKGROUND OF THE INVENTION

Breast pumps are used by breast feeding women to extract milk from their breast such that the extracted milk can be fed to their babies at a later time. Typically, the breast is placed into a funnel shaped cup and a vacuum is applied such that milk is extracted. The breast pump typically has two operating modes, namely a stimulation mode and an extraction mode. During the stimulation mode, the milk ejection reflex is stimulated. When the user is only placing one breast into the breast cup, the user can use the other hand to start or activate the breast pump. However, if the user is placing both their breasts into the breast cups, the user does not have any hand free for activating the breast pump. When the breast pump is activated, a pressure source like a vacuum pump inside the breast pump will generate a vacuum and the stimulation mode can be started.

It is, however, sometimes difficult for the user to correctly position the breast inside the breast receiving funnel of the breast pump before activating the breast pump. If two hands are required to hold the breast receiving funnel then it can be difficult to activate the pressure source. On the other hand, if the pressure source is activated at first than it may be difficult to position the breast receiving or funnel.

US 2011/004154 A1 describes a breast pump having a sensor for measuring a parameter of the pump during the use of the breast pump. The sensor can be a pressure sensor or a milk flow sensor. The pressure sensor is adapted to measure a negative pressure in a breast cup. The milk flow sensor is adapted to measure a milk flow. If the pressure measured by the pressure sensor differs from a predetermined pressure, a controller can adjust the settings of the breast pump.

WO 2017/208243 A1 discloses a breast pump with air pumps and a processing system connected to the pumps. The breast pump furthermore comprises a user interface for user actuation of the air pumps. A placement of a breast into an expression kit can be detected.

U.S. Pat. No. 7,396,340 B2 discloses a breast pump with a power source and a breast receiving portion.

EP 2 575 919 A1 discloses a breast pump device having breast receiving portions, a power source and a controller to control the operation of the pressure source. Furthermore, the presence of a breast in the breast receiving portion can be detected.

### SUMMARY OF THE INVENTION

It is therefore an object of the invention to provide a breast pump device which improves the usability of the breast pump at the beginning of the expression session.

This object is solved by a breast pump device which has at least one breast receiving portion for receiving a breast of a user. The breast pump device furthermore comprises a pressure source coupled to the at least one breast pump receiving portion. The pressure source is capable of creating an overpressure or an underpressure (vacuum) when the breast is placed into the at least one breast receiving portion. The pressure source can be operated in a variety of operating modes, for example a first operating mode (e.g. a detection mode or an off mode) and a second operating mode for milk extraction corresponding e.g. to a stimulation mode or an extraction mode. The breast pump device furthermore comprises a controller for controlling the operation of the pressure source. The breast pump device furthermore comprises a breast detector coupled to the controller. The breast detector detects a first state when no breast is placed in the at least one breast receiving portion or a second state when the breast is placed into the at least one breast receiving portion based on operating parameters of the pressure source like a vacuum source. The breast detector detects the first or second state based on operating parameters of the pressure source. The breast detector communicates the presence of the first or second state to the controller. The controller may initiate an action based on the detection of the first or second state by the breast detector. The controller operates the pressure source in a first operating mode if the breast detector has detected the first state or in the second operating mode if the breast detector has detected the second state. Hence, the controller of the breast pump can take an action when the breast has been detected in the breast receiving portion.

Based on the detection of the first state (no breast in breast receiving portion) or the second state (breast in breast receiving portion), the controller activates a first operating mode (detection mode) or a second operating mode (milk extraction).

Hence, a breast pump is provided which enables an automotive breast detection and an automotive start of the expression cycles during milk extraction.

According to an embodiment of the invention the action taken by the controller can be a visual or audible indication of the state, a change in an operating parameter of the device or the storage of the information of the state-change. The controller can be configured to change a parameter of the breast pump device and/or to output a notification if the breast detector has detected the second state. Such a notification may also include a signal or command or an entry in a log or in a memory.

According to an embodiment of the invention, the operating parameters of the pressure source can be the speed or acceleration of a motor of the pressure source, voltage or current measurements at the pressure source or the underpressure or overpressure created by the pressure source.

According to an embodiment of the invention, the operating parameters can be analyzed over time.

According to an embodiment of the invention, the operating parameters of the pressure source are chosen in such a way that the presence of a breast causes a detectable change in at least one parameter measurable by the breast detector.

According to an embodiment of the invention, the breast detector can also take into account leakage which occurs when the breast is placed into the breast receiving portion and an overpressure or underpressure is applied.

According to an embodiment of the invention, when a breast pump is activated, reference measurements of the operating parameters of the vacuum source can be conducted.

According to an embodiment of the invention, the controller can control the vacuum source in an initial placement mode where the vacuum source applies a low vacuum such that the expression kit (the breast receiving portion) can be easily positioned over the breast thus enabling a detection mode for the detection of a breast in the breast receiving portion.

According to a further embodiment of the invention, if the breast detector does not detect the second state, in response to a user input or in response to an external or internal signal like a timer, the controller can initiate the second operating mode in order to start the stimulation or extraction mode even if the second state has not yet been detected. The stimulation mode and/or the extraction mode can also be a sequence of different operating modes in order to extract milk.

According to a further embodiment of the invention, the controller can operate a second pressure source based on the input from a detector for a first pressure source.

According to an embodiment of the invention, the controller operates the pressure source in a detection mode as the first mode, wherein the pressure source generates a low vacuum. The controller is configured to operate the pressure source in the second operating mode for milk expression. The second operating mode may be divided into submodes.

According to an embodiment of the invention the controller initiates the second operating mode of the pressure source based on an input from the user, based on an external signal or if the breast detector has not detected the second state after a predefined period of time.

According to an embodiment of the invention the breast pump comprises at least one breast receiving portion for receive a breast of a user, a pressure source coupled to the at least one breast receiving portion and being configured to generate an overpressure or an underpressure according to at least a first operating mode or a second operating mode, a controller configured to control an operation of the pressure source according to the first or second operating mode, a breast detector coupled to the controller and being configured to detect a first state when no breast is placed in the at least one breast receiving portion and to detect a second state when the breast is placed into the at least one breast receiving portion or to detect a third state when the breast is not placed correctly into the breast receiving portion such that leakage occurs based on operating parameters of the pressure source and to communicate the third state to the controller. The third state can also relate to operating parameters which deviate from operating parameters of a normal operation. The controller is configured to operate the pressure source in a first operating mode if the first state has been detected or a second operating mode if the second state has been detected. The controller is configured to operate the pressure source in a third operating mode if the third state has been detected.

According to an embodiment of the invention the controller changes a parameter of the breast pump device and/or outputs a notification, signal or command if the breast detector has detected the third state.

According to an embodiment of the invention the breast detector is configured to detect the third state based on operating parameters of the pressure source.

According to an embodiment of the invention the controller is configured to initiate a different sequence of modes or program than the original mode or sequence of modes after an interruption.

According to an embodiment of the invention the pressure source can be implemented as a hydraulic pump or as an electrical pump.

According to an embodiment of the invention the breast receiving portion is configured to be mechanically movable or flexible such that the breast inside the breast receiving portion can be massaged to improve the milk extraction.

In addition to the first and second operating mode, the breast pump device may comprise a further operating mode being a calibration mode during which operating parameters of the pressure source can be detected and be calibrated.

The breast pump device can perform an automatic breast detection based on operating parameters of the pressure source of the breast detector. A first state is detected when no breast is placed in the breast receiving portion and a second state is detected when a breast is placed into the breast receiving portion. During a first operating mode, which corresponds to the detection mode, the breast detector monitors the operating parameters of the pressure source. During the first operating mode, optionally a low over- or underpressure can be applied such that the breast can still be moved in the breast receiving portion to enable the user a comfortable fit of the breast in the breast receiving portion. Once the breast is placed in the breast receiving portion and the second state is detected by the breast detector, the second operating mode can be activated to initiate a milk extraction. If, however, the breast is not placed correctly in the breast receiving portion and a leakage is detected (for example because the over- or underpressure in the breast receiving portion does not correspond to the expected over- or underpressure or if the measured operating parameters do not correspond to typical values of the operating parameters), then the controller can operate the power source in a third operating mode to enable a correct placement of the breast in the breast receiving portion. With such a breast pump device, an automatic breast detection can be performed and the milk extraction can be performed without any additional input of the user. Thus, the user only needs to switch on the breast pump device to place the breast in the breast receiving portion. The other sequence of events will be performed by the breast pump device.

Further aspects of the invention are defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 discloses a block diagram of a breast pump device according to an embodiment of the invention,

FIG. 2 shows a flow chart of the operation of the breast pump device according to an embodiment of the invention,

FIG. 3 shows a schematic representation of a breast pump device according to an embodiment of the invention,

FIG. 4 shows a graph depicting a behavior of a vacuum pump according to an embodiment of the invention,

FIG. 5 shows a graph indicating a current profile for a motor of the pressure source of a breast pump,

FIG. 6 shows a current profile of an electrical motor of a breast pump,

FIG. 7 shows a current profile of an electric motor of a breast pump,

FIG. 8 shows a current profile of a motor for a breast pump, and

FIG. 9 shows a current profile of a motor of a breast pump.

### DETAILED DESCRIPTION OF EMBODIMENTS

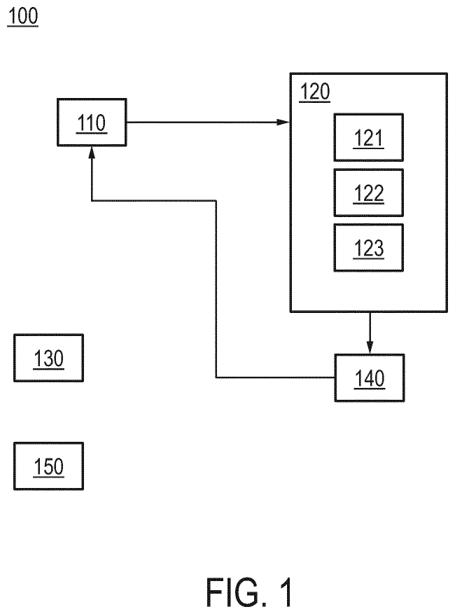

FIG. 1 discloses a block diagram of a breast pump device according to an embodiment of the invention. The breast pump device 100 comprises a controller 110, a pressure source 120 configured to generate an overpressure or an underpressure (vacuum source) 120, at least one breast receiving portion 130, a breast detector 140 and a milk extraction path 150. The pressure source 120 can comprise a motor 121 for creating an overpressure or an underpressure (e.g. a vacuum). The pressure source 120 may optionally comprise a sensor capable of measuring a characteristic of the pressure sources such as voltage or current or pressure and flow. Such a sensor can be a speed sensor 122 and a voltage or current sensor 123. The speed sensor 122 can be used to detect the speed of a motor 121 of the pressure source 120. The voltage or current sensor 122 can be used to detect the voltage or current of the motor 121.

According to an embodiment of the invention, the pressure source 120 is implemented as a vacuum source generating an underpressure.

The breast detector 140 is used to detect whether a breast is placed into one of the breast receiving portions 130. If the breast detector 140 does not detect a breast inside the breast receiving portion 130, the breast detector 140 will indicate a first state (no breast inside the breast receiving portion 130) to the controller 110. When the breast detector 140 detects the presence of a breast in one of the breast receiving portions 130, the breast detector 130 can communicate the second state to the controller 110.

According to an embodiment of the invention, the breast detector 140 uses operating parameters of the pressure source 120, like a vacuum source in order to detect the presence of a breast in the breast receiving portion 130. Hence, the breast detector 140 can differentiate between a first state (no breast) and a second state (breast in the breast receiving portion).

The operating parameters of the pressure source, like a vacuum source 120 can be the speed or acceleration of the motor 121 or the voltage or current of the motor 121.

The controller 110 will activate the second operating mode (stimulation mode) of the pressure source, like a vacuum source 120 when the breast detector 140 has indicated the second state. However, if the breast detector 140 does not indicate the second state but the first state, the controller 110 will not initiate the second operating mode of the pressure source, like a vacuum source 120.

Optionally, the pressure source, like a vacuum source 120 can have a (breast) detection mode as first operating mode. The (breast) detection mode may be activated by the controller 110 when the breast pump 100 is switched on. In the detection mode, the pressure source, like a vacuum source 120 can be optionally used to create a low vacuum such that the user can place the breast into the breast receiving portion without having the difficulties of coping with a high vacuum which can make the movement of the breast inside the breast receiving portion 130 difficult. In other words, during the detection mode, a low vacuum is applied by the pressure source, like a vacuum source to enable a comfortable placement of the breast inside the breast receiving portion. Alternatively, the change to a second operating mode as initiated when second state is detected can be delayed to allow additional time for a user to properly position the breast into the breast receiving portion.

The breast pump 100 can have a milk extraction path 150 which can include the breast receiving portion 140 (expression kit or breast cup). The milk extraction path 150 is the path the extracted milk follows until it flows into a bottle or vessel which can be attached to the breast pump device. The breast receiving portion 130 can be funnel shaped.

The controller 110 can activate the second operating mode for milk expression (first a stimulation mode and extracting mode) when the breast detector 130 has detected the presence of a breast in the breast receiving portion 130 (i.e. the second state). Thereafter, in the second operating mode, an extracting mode can be initiated by the controller 110 in order to extract milk from the breast of a user.

According to an embodiment of the invention, the breast detector 140 can be implemented as a dedicated unit. Alternatively, the controller 110 can perform at least some of the functions of the breast detector 140.

The pressure source 120 can be implemented as an electrical pump or as a hydraulic pump. Optionally, the breast receiving portion 130 is configured to be mechanically movable or flexible such that the breast inside the breast receiving portion can be massaged to improve the milk extraction.

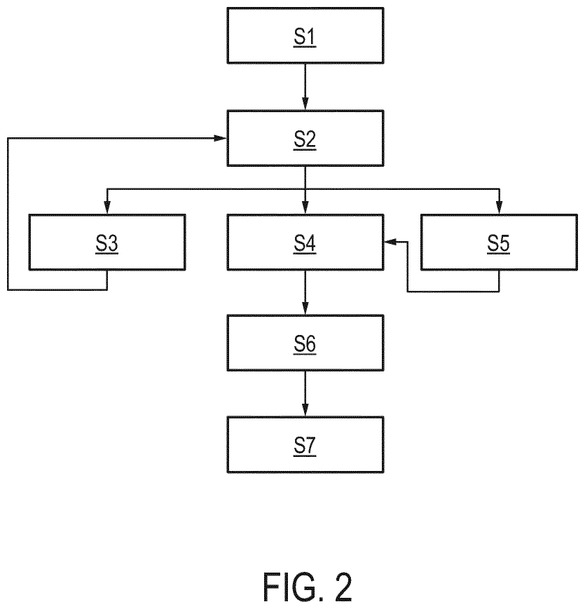

FIG. 2 shows a flow chart of the operation of the breast pump device according to an embodiment of the invention. The breast pump device according to FIG. 2 can correspond to the breast pump device according to FIG. 1. In step S1, the breast pump 100 is in the off-state. In step S2, the breast pump 100 is switched on and a test (detection mode) is initiated. In other words, the breast detector 140 detects whether a breast is present in the breast receiving portion 130. Hence, the controller 110 may initiate the detection mode. If no breast has been detected in the breast receiving portion 130 by the breast detector 140, the flow continues to step S3, returning the flow to step S2 to perform another test (by initiating the detection mode). If, however, a breast is detected in the breast receiving portion 130 (second state, step S4), the milk expression sequence (second operating mode of the vacuum source) is initiated in step S6.

The milk expression sequence can comprise a stimulation mode followed by an expression mode. If after a predetermined period of time the breast detector has not detected a breast in the breast receiving portion 130, then the controller can still initiate the second operating mode. Alternatively, the controller 110 may initiate the second operating mode based on an input of the user or based on an external signal even if no breast has been detected. In step S7, the vacuum can be reduced by the pressure source, like a vacuum source 120 and the breast pump 100 can be deactivated as the milk expression has ended.

If after a predefined time period the breast detector is still detecting the second state, the flow can continue to step S4 and the controller 110 can activate the second operating mode of the pressure source, like a vacuum source 120 to start the milk expression.

Optionally, the breast detection can be performed for each breast which is placed in a breast receiving portion (i.e. an expression kit). Accordingly, the breast pump device can improve the flexibility of the milk extraction. For example, when the left breast is already correctly placed into a breast receiving portion, the controller can initiate a second operating mode for the left breast while the right breast is still being placed correctly into the breast receiving portion. Alternatively, the controller 110 may only initiate the second operating mode when the breast detector has detected that both breasts are placed correctly in the breast receiving portion. Alternatively, if a user only wants to extract milk from one breast, then the controller 110 may detect that the other breast is not placed into the breast receiving portion and still can activate the second operating mode for milk extraction.

One or two expression kits can be provided each with breast receiving portions (breast cup) into which a breast can be placed. Hence, milk extraction can optionally be performed for each breast separately. Alternatively, the milk extraction can be performed for both breasts.

According to an embodiment of the invention, the breast detector 140 uses operating parameters of the pressure source, like a vacuum source to determine a first or second state, namely whether or not a breast is placed into the breast receiving portion 130. The operating parameter can be the voltage or current of the pressure source, like a vacuum source 120 and in particular those of a motor 121 of the pressure source 120 like a vacuum source. The controller 110 can initiate the second operating mode (stimulating mode or the expression mode of the pressure source, like a vacuum source 120) if the breast detector 140 has detected a breast in one of the breast receiving portions 130. When after a certain time period no breast is detected in the other breast receiving portion, the controller 110 can switch from stimulation to expression mode or vice versa (both second operating mode). Alternatively, the controller 110 may switch from the second operating mode to the first operating mode.

The breast detector 140 can optionally take leakage of vacuum into account (for example as the breast may not be exactly positioned inside the breast receiving portion).

According to an embodiment of the invention, the detecting mode of the pressure source, like a vacuum source 120 can be initiated by the controller 110 when the breast pump is activated. The detection mode can be used until a breast is detected inside the breast receiving portion 130. In addition, the detection mode can also be used after a breast has been detected in the breast receiving portion (e.g. at intervals) as an in-between measurement during the second operating mode in order to determine whether the breast is still inside the breast receiving portion or to determine whether the breast is placed into the breast receiving portion and that there is no or little leakage.

According to an embodiment of the invention, the breast detector 140 may operate continuously or at intervals. The breast detector 140 can also determine a leakage as well as a pausing of the pressure source, like a vacuum source. This information can be notified to the user via the breast pump device or via an external device like a smart phone.

When the breast pump is activated, optionally a measurement of the operating parameters of the pressure source, like a vacuum source can be performed. These measurements can be used as reference values.

According to an embodiment of the invention, the detector 140 may be coupled to a vacuum sensor to detect the vacuum created by the pressure source, like a vacuum source 120. According to an embodiment of the invention, after the detection of the presence of a breast in the breast receiving portion 130, the pressure source, like a vacuum source 120 may be operated at various operating modes by the controller 110. In particular, various suction profiles can be implemented by the pressure source, like a vacuum source 120. These suction profiles may be adapted over time.

During the detection mode, the controller 110 controls the pressure source, like a vacuum source 120 to generate a low vacuum. This is in particular advantageous as it allows the user to correctly place the breast inside the breast receiving portion 120. Once the breast detector 140 has detected the correct placement of the breast in the breast receiving portion, the controller 110 may initiate a stimulation mode of the pressure source, like a vacuum source 120.

According to an embodiment of the invention, the controller 110 may initiate the second operating mode (stimulation mode or expression mode of the vacuum source 120) even if the breast detector 140 has not detected the correct placement of a breast inside the breast receiving portion 130 after a predefined period of time. This is advantageous as it will allow the operation of the breast pump to extract milk even if the breast detector has not yet detected the correct placement of a breast inside the breast receiving portion. In other words, this feature allows a fallback operation of the breast pump.

According to an embodiment of the invention, the breast pump may comprise massaging expression kits which allow a massaging of the breast during the stimulation and extraction mode of the vacuum source.

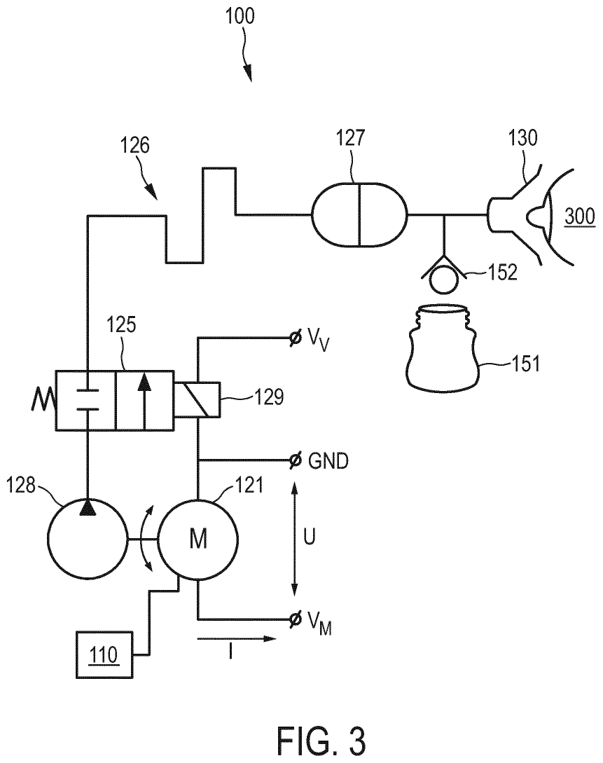

FIG. 3 shows a schematic representation of a breast pump device according to an embodiment of the invention. The breast pump device 100 according to the invention is an electric breast pump which comprises a funnel 130 as breast receiving portion, a motor 121 for driving a vacuum pump 128 as well as a release valve 125. The vacuum pump 128 is coupled to a membrane 127 via a tube 126. Moreover, a solenoid 129 is provided to drive the valve 125. A breast 300 of a user is placed into the funnel 130 (breast receiving portion) and the motor 121 is activated in order to create a vacuum in the funnel 130. The valve 152 is provided allowing air and milk to flow towards a milk storage container or milk bottle 151. The controller 110 controls the voltage of the motor 121 and the solenoid. The current (I) and/or the voltage (U) at the motor 121 can be detected.

The power of the motor 121 is calculated by a multiplication of the current and the voltage (P=U×I). If a load is present for the pump 128 then the amount of power which is required by the motor 121 will increase. If either the current or the voltage of the motor 121 is fixed, the presence of a load will result in an increase of the other parameter. If either the current or the voltage is set at a low value, the increase of the other parameter will be more pronounced and thus easily detectable.

By detecting the increase of one of the parameters (current, voltage), the breast pump device can detect whether or not a breast has been placed into the funnel or the breast receiving portion. Accordingly, an automatic detection of the presence of a breast in the breast receiving portion is achieved. Hence, the breast detection is performed based on the monitoring of operation parameters of the pressure source, like a vacuum source 120.

According to an embodiment of the invention, the motor 121 can be operated in the region of a stall voltage of the motor. If for example the breast 300 is then placed into the funnel 130, then a load detection of the breast pump device can be performed. In order for the load detection, it is only required to monitor the current flowing through the motor 121. If a breast 300 is placed into the funnel 130, this will lead to a significant increase of the current of the motor 121. This significant current increase can be detected and the presence of a breast in the funnel or the breast receiving portion can be thus detected.

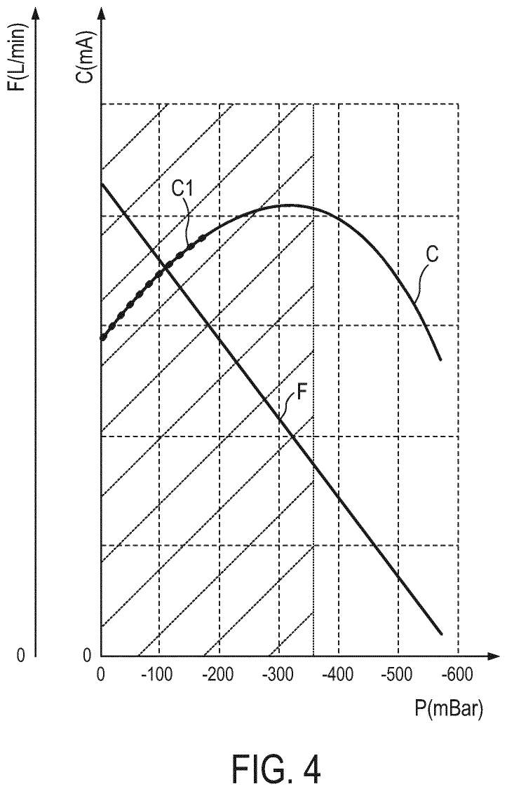

FIG. 4 shows a graph depicting a behavior of a vacuum pump according to an embodiment of the invention. In FIG. 4, the pressure P, the current C and the flow F are depicted. FIG. 4 in particular discloses the pump behavior in relation to the achieved pressure. As can be seen, with increasing pressure P, the flow F increases. Furthermore, with increasing pressure P, the current C will increase until a maximum and will then decrease again. According to an embodiment of the invention, optionally only the first portion C1 of the current function C is used as this first portion C1 is substantially linear and therefore predictable. The breast detector 140 can be implemented e.g. by a current measurement or power measurement of the motor as described above.

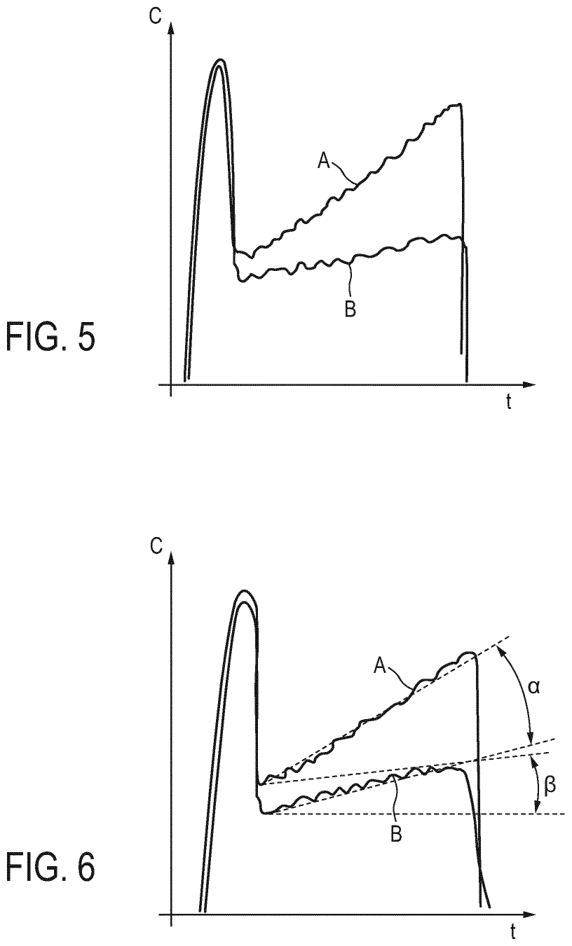

FIG. 5 shows a graph indicating a current profile for a motor of the pressure source of a breast pump. In FIG. 5, a graph of a current profile of the motor 121 of the breast pump is shown with a breast inside the breast receiving portion (profile A) and without a breast inside the breast receiving portion (profile B). In this embodiment, the voltage applied to the motor 121 is kept substantially constant such that the current increases a load (breast inside the breast receiving portion; current profile A) is present. According to the embodiment of FIG. 5, the difference between the current profile A (with a breast) and the current profile B (without a breast) can be determined by the breast detector 140. Accordingly, the breast detector 140 can determine whether or not a breast is present in the breast receiving portion 130 by measuring the current flowing through the motor 121 and comparing this current with a reference current associated to the current profile B (without a breast).

FIG. 6 shows a current profile of an electrical motor of a breast pump. In FIG. 6, the current profile A of a breast pump with a breast inside the breast receiving portion 130 and a current profile B of a breast pump without a breast in the breast receiving portion is depicted. FIG. 6 also shows an angular difference over time α for the current profile A and an angular difference over time β for the current profile B. Hence, the breast detector 140 can monitor a slope of the current profile A of the motor 121 and compare it to a reference slope of the motor without a breast in the breast receiving portion. In other words, the breast detector 140 can use a detected angular difference to determine whether or not a breast is present in the breast receiving portion.

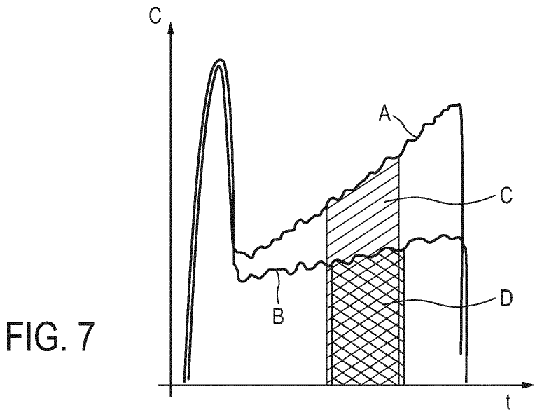

FIG. 7 shows a current profile of an electric motor of a breast pump. In FIG. 7, in particular a current profile A of a motor 121 of the breast pump is shown if a breast is inside the breast receiving portion. The current profile B corresponds to the current profile of the motor 121 when no breast is present in the breast receiving portion. The breast detector 140 can determine a used energy of a motor 121 of the breast pump when a breast is placed inside the breast receiving portion. Furthermore, the breast detector can compare this used energy with a reference used energy D related to a current profile of a motor when no breast is placed in the breast receiving portion. By comparing the two used energies C, D, the breast detector 140 can determine whether or not a breast is present in the breast receiving portion.

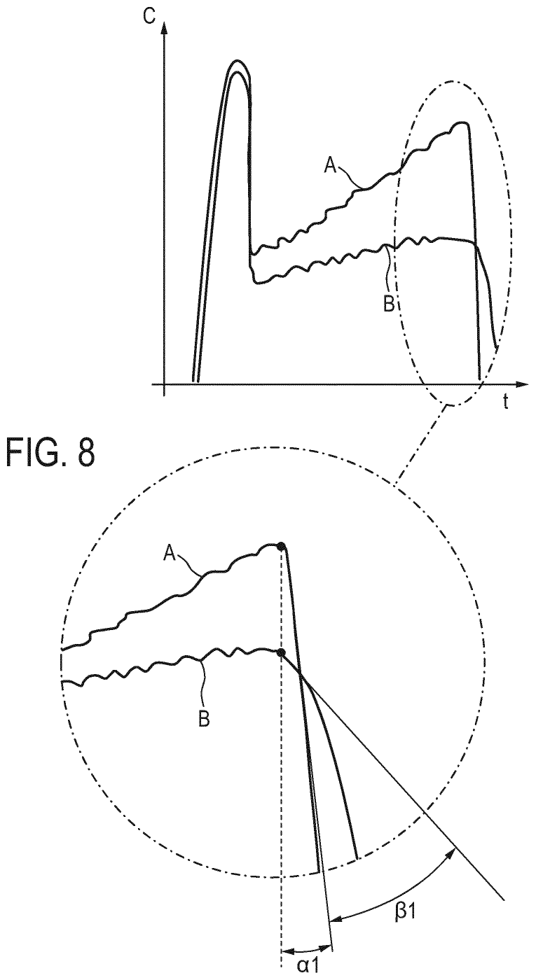

FIG. 8 shows a current profile of a motor for a breast pump. In particular, in FIG. 8, a current profile A for the motor 121 with a breast placed in the breast receiving portion is depicted. Furthermore, a current profile B of a motor 121 of the breast pump 100 corresponds to the current profile when no breast is placed inside the breast receiving portion. According to FIG. 8, the breast detector 140 can compare a time difference between the current profile A and the current profile B to determine whether or not a breast is placed inside the breast receiving portion 130.

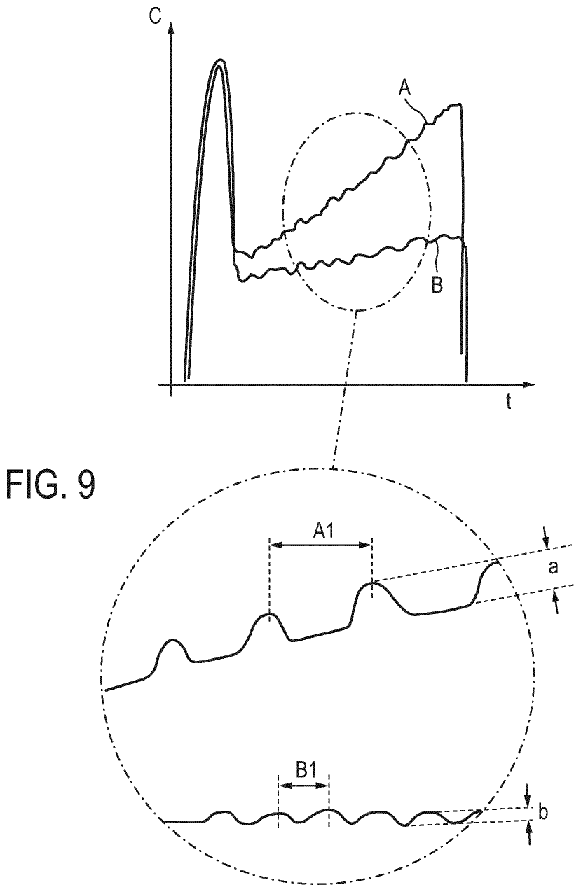

FIG. 9 shows a current profile of a motor of a breast pump. In FIG. 9, the current profile A (with a breast) and the current profile B (without a breast) is depicted. FIG. 9 also shows an enlarged section of the current profiles A, B. The motor 121 may superimpose a ripple on the drawn current. The ripple can be indicative of the speed of the motor. The current profile A has ripples with a height a and a distance A1 between two ripples. The current profile B also has a ripple height b and a distance B1 between two ripples. The breast detector may detect the ripples and in particular the height of the ripples and the distance between two ripples in order to determine a speed of the motor. The detected speed of the motor can then be used to determine whether or not a breast is present in the breast receiving portion.

According to a further embodiment of the invention, the breast detector may also use parameters other than the operating parameters of the pressure source, like a vacuum source to detect the presence of a breast. Hence, the motor rotation may be used by the breast detector. Alternatively, the breast detector may comprise a pressure sensor, a flow sensor, a capacitive sensor, a resistive sensor, an optical sensor, a mechanical sensor, a chemical sensor, a temperature sensor, an acoustic sensor, a magnetic sensor and/or a vital body signs sensor. Furthermore, the breast detector 140 may use a detection of human body in the breast receiving portion 130.

According to an embodiment of the invention, in a detection mode, the breast pump device may be operating in a stall running mode, i.e. the motor is operated in the region of the stall voltage such that the speed of the motor is reduced.

The breast pump according to the invention can be worked for open or closed systems (with or without balg or membrane 127). Accordingly, the breast pump can still be operated if the valve 125 and/or tube 126 are removed.

According to an embodiment, the breast detector detects a first state when no breast is placed in the at least one breast receiving portion or a second state when the breast is placed into the at least one breast receiving portion based on operating parameters of the pressure source.

To let the controller identify the difference between a threshold operating parameters and the actual (measured) operating parameters, the controller can be controlled with a fixed threshold operating parameter. In an alternative scenario a flexible threshold operating parameter can be used. This flexible threshold operating parameter, could be based on a fixed evolution of values over time (e.g. based on known life-time evolution of operating parameters) or could be based on a calibration measurement (measure operating parameters when no breast is present and storing them as new threshold operating parameters).

For the calibration method, it must be ensured that no breast is present or influencing the operating parameters (measured) during calibration. This can be achieved this by instructing the user. This could also be performed by using existing components in the breast pump. Especially the atmosphere release valve 125 could be used for this. During typical breast pump operation over or under pressure is created by the pressure source, after a period of time pressure is released to atmosphere. This release to atmosphere is created by opening the atmosphere release valve 125. During calibration we could use this valve to remove the influence of the breast to the operating parameters. Hence, this valve is left open so that when the pressure source creates over or under pressure it runs freely (without effect of a breast being present) even if a breast is present in the breast receiving portion.

The pressure source 120 can be implemented as an electrical pump or as a mechanical balg or any other device that can generate an underpressure or an overpressure which can be used for milk extraction.

According to an embodiment, if the first state (no breast) has been detected by the breast detector, the controller 110 may activate a power saving mode or may reduce the power consumption of the breast pump. This allows a reduction in power consumption, can save breast pump life time and can reduce the heat production.

According to an embodiment, the pump can be integrated into the expression kit. The pump may comprise a motor which is capable of moving a diaphragm (directly) near a nipple of a user creating an under pressure.

According to an embodiment an under pressure storage is provided which is able to supply an under pressure. The storage may be controlled by valves. Parameters of the valves can be used as indicators for the presence of a breast.

According to an embodiment the breast pump device can comprise multiple pumps, multiple motors and/or multiple expression kits.

Other variations of the disclosed embodiment can be understood and effected by those skilled in the art in practicing the claimed invention from a study of the drawings, the disclosure and the appended claims.

In the claims, the word “comprising” does not exclude other elements or steps and in the indefinite article “a” or “an” does not exclude a plurality.

A single unit or device may fulfill the functions of several items recited in the claims. The mere fact that certain measures are recited in mutual different dependent claims does not indicate that a combination of these measurements cannot be used to advantage. A computer program may be stored/distributed on a suitable medium such as an optical storage medium or a solid state medium, supplied together with or as a part of other hardware, but may also be distributed in other forms such as via the internet or other wired or wireless telecommunication systems.

Any reference signs in the claims should not be construed as limiting the scope.

1. A breast pump device, comprising
at least one breast receiving portion configured to receive a breast of a user,
a pressure source coupled to the at least one breast receiving portion and being configured to generate an overpressure or an underpressure according to at least a first operating mode which is a breast detection mode or a second operating mode which is a milk expression mode,
a controller configured to control an operation of the pressure source in the first operating mode or in the second operating mode, and
a breast detector coupled to the controller and being configured to detect a first state when no breast is placed in the at least one breast receiving portion and to detect a second state when the breast is placed into the at least one breast receiving portion and to communicate the first or second state to the controller,
wherein the breast detector is configured to detect the first or second state based on operating parameters of the pressure source,
wherein the controller is configured to operate the pressure source in the first operating mode while the breast detector has detected the first state or in the second operating mode once the breast detector has detected the second state.

2. The breast pump device according to claim 1, wherein the controller is configured in the second operating mode to change a parameter of the breast pump device or to output at least one of a notification, a signal and a command, if the breast detector has detected the second state.

3. The breast pump device according to claim 1, wherein the operating parameters of the pressure source are selected in such a way that a presence of a breast causes a detectable change in the operating parameter of the pressure source.

4. The breast pump device according to claim 1, wherein the operating parameters of the pressure source include speed or acceleration data of a motor of the pressure source or voltage or current measurements of the motor.

5. The breast pump device according to claim 1, wherein in the first operating mode, the pressure source is configured to generate a low underpressure allowing movement of the breast inside the breast receiving portion.

6. The breast pump device according to claim 1, wherein the controller is configured to initiate the second operating mode of the pressure source based on an input from the user, based on an external signal or if the breast detector has not detected the second state after a predefined period of time.

7. A breast pump device according to claims 1,
wherein the breast detector is configured to detect a third state when the breast is not placed correctly into the breast receiving portion such that leakage occurs based on operating parameters of the pressure source or if the operating parameters of the pressure source deviate from operating parameters of a normal operation of the power source and to communicate the third state to the controller,
wherein the controller is configured to operate the pressure source in a third operating mode if the third state has been detected.

8. The breast pump device according to claim 7, wherein the breast detector is configured to detect the third state based on operating parameters of the pressure source.

9. The breast pump device according to claim 1, wherein the controller is configured to initiate a different sequence of modes or program than the original mode or sequence of modes after an interruption.

10. The breast pump device according to claim 1, wherein
the controller is configured to initiate a calibration mode for calibrating operating parameters of the power source.

11. The breast pump device according to claim 1, wherein
the controller is configured to initiate a calibration mode for calibrating operational parameters of the power source.

12. A non-therapeutic method of operating a breast pump device which has at least one breast receiving portion, a pressure source, a controller and a breast detector, comprising the steps of
in a first operating mode which is a breast detection mode, detecting whether a breast is placed into the breast receiving portion by the breast detector and communicating a first state to the controller when no breast is placed in the breast receiving portion or a second state when the breast has been detected inside the breast receiving portion,
controlling an operation of the pressure source in the first operating mode while the breast detector has detected the first state or in a second operating mode which is a milk expression mode once the breast detector has detected the second state by the controller,
detecting the first or second state based on operating parameters of the pressure source by the breast detector,
changing a parameter of the breast pump device and/or outputting a notification, signal or command, if the breast detector has detected the second state.

13. (canceled)
